(19) United States
(12) Patent Application Publication
(10) Pub. No.: US 2023/0394906 A1
(43) Pub. Date: Dec. 7, 2023

(54) Vehicle Mini-bar With Touchless Interface

(71) Applicant: International Automotive Components Group GmbH, Düsseldorf (DE)

(72) Inventors: Carter Scott CANNON, München (DE); Sung-uk KIM, Ebersberg (DE)

(21) Appl. No.: 18/246,392

(22) PCT Filed: Sep. 24, 2021
(86) PCT No.: PCT/EP2021/076402

§ 371 Date: Mar. 31, 2023

Related U.S. Application Data
(60) Provisional application No. 63/083,261, filed on Sep. 25, 2020.

(51) Int. Cl. G07F 13/10

(52) U.S. Cl. CPC G07F 13/10; G06Q 20/18

(57) ABSTRACT

A vehicle mini-bar comprises a housing containing one or more items therein for dispensing; an interface configured to detect and allow one or more users to access and purchase a user selected item located within said housing; one or more cameras and sensors to identify one or more items contained in said housing; one or more sensors to identify a selected user; a processor with recognition software in communication with a user device (APP) wherein said processor reports to said user device the selection and purchase by a user of said user selected item; and wherein said mini-bar includes one or more UVC light emitting diodes (LEDs) and said processor is configured to activate said one or more UVC LEDs to implement a disinfecting lighting sequence.

### FIELD

The present invention relates to a vehicle mini-bar preferably with a touchless interface including the ability to determine and assign a dispensing activity to a selected purchaser and to apply a UVC lighting disinfecting protocol.

### BACKGROUND

Mini-bars are found in various hospitality establishments and can be stocked with food, beverage and/or convenience items for the consumer. Various efforts have been reported to provide for, what is sometimes termed a vending device within a vehicle, or what may be identified as a mobile-vending/kiosk apparatus or system. Along such lines, reference is made to U.S. Pat. Nos. 5,884,957, 6,234,345; 7,737,855; 8,140,185; 10,124,708; 9,741,014; 10,002,486; 10,134,218 and U.S. Publication Nos. 2006/0049198, 20040249502, 20080055058, 20090289109; 20110136429.

A need remains, however, particularly in light of current worldwide pandemic considerations, to provide a vehicle mini-bar that preferably offers a touchless interface, the ability to assign dispensing activity to a selected purchaser as well as the ability to select and implement a UVC lighting disinfecting protocol.

### SUMMARY

The present invention relates to mini-bar preferably with a touchless interface, for placement in a vehicle that is configured to dispense, e.g., a beverage, food item, and/or consumer convenience items. It is contemplated that such mini-bar would therefore be of particular benefit in a variety of vehicular environments, including but not limited to placement in passenger vehicles, e.g. automobiles, robo-taxis, ride-share operations, as well as placement in a train, airplane, bus, boat, etc. While the mini-bar herein is contemplated to be a stand-alone unit, it should be understood that the mini-bar may also be configured to position within or exist as part of a vehicle trim component. Moreover, while the mini-bar is preferably one with a touchless interface, it is contemplated herein that the interface may be one that allows the user to manually unlock and open the mini-bar, or one that includes a touch sensor that identifies the user and then open the mini-bar for item dispensing. In these optional embodiments, all of the other features of the mini-bar remain present, such as the ability of the mini-bar system to identify a user and to automatically identify and bill/invoice items that are dispensed, and to implement any sterilization protocol.

The items that may be included into the mini-bar are not limited. That is, such items may include any of those items that a consumer may find of need when in a vehicle and travelling to a desired location. Such items may include, but not be limited to, beverages or food products, and in particular beverages and/or food products that are initially provided in cooled condition. Furthermore, as described further herein, the selected items may also be further cooled or heated by incorporation of a unit on the exterior of the mini-bar that provides the option to further heat/cool, as desired. In such manner, the consumer herein can be assured that their selected item may be generally maintained in a cooled or heated state through-out their respective ride experience.

As alluded to above, the items contemplated for dispensing herein from the mini-bar include food items that may be maintained at an initial desired cooled condition. The food items may also include non-perishable food items that do not require temperature regulation. Such non-perishable food items may include, e.g., what may be generally known as dry goods, not requiring refrigeration, such as potato chips, fruit (orange/apple), dried fruit, cereal, granola and nuts.

The items contemplated for dispensing may also include consumer convenience items that users may typically find in need while traveling. Such may include, but are not limited to, hand-sanitizer, disinfectant wipes, toiletries (toothpaste, mouthwash, combs/brushes), charging devices/cords, headphones, etc.

### SHORT DESCRIPTION OF DRAWINGS

FIG. 1 identifies in block diagram format some of the features of the mini-bar system herein.

FIGS. 2-5 show in sequence a preferred example of a user accessing and utilizing the mini-bar.

FIG. 6 illustrates in cut-away view a preferred configuration for construction of the vehicle mini-bar herein.

FIG. 7 illustrates the versatility contemplated for the mini-bar herein in cross-section.

### DETAILED DESCRIPTION OF EXAMPLES

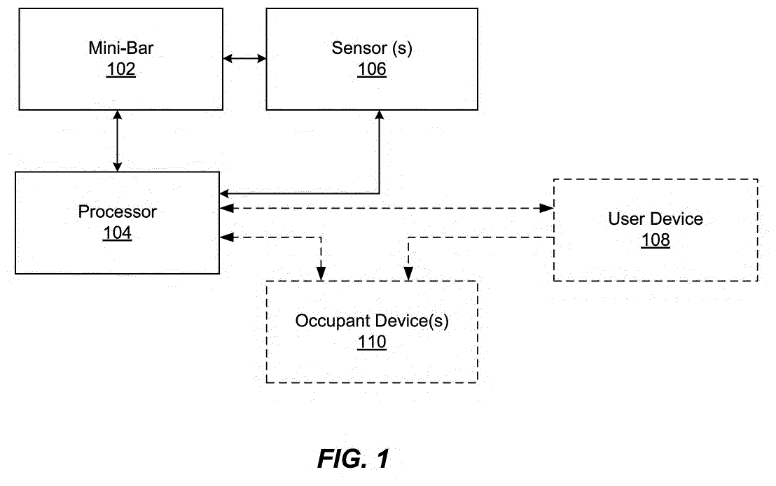

Reference is now made to FIG. 1 which identifies in block diagram format some of the features of the mini-bar system herein. The mini-bar at 102 is one that includes a processor 104 and one or more sensors 106. Sensors 106 may preferably include one or more camera sensors with vision recognition software which can image, identify the selected item and report to the processor which vehicle occupant's account, among those present, has decided to utilize the mini-bar to select, remove and purchase one or more of its contents. The processor 104 is configured to be in wireless communication (WiFi/Bluetooth/NFC) with one or more devices, such as a user device 108 or occupant device 110 either of which may include an application (APP) to initiate, monitor and record any dispensing activity of the mini-bar. As may therefore be appreciated, when a vehicle occupant therefore decides to select from the mini-bar a particular item, the processor 104 along with the one or more camera sensors and vision recognition software can now confirm the particular individual who is then accessing the mini-bar and report to a user device 108 or occupant device 110 on the particular item selected item for purchase, and the occupant device 110 can then be notified of a charge/invoice to the occupant in an appropriate amount for the sale of the selected item.

Alternatively, the processor may utilize biometric recognition software to identify one or more identifying characteristics of an individual, e.g. fingerprint scan, voice recognition, retina recognition, heart rate scan, etc. However, in the broad context of the present invention, it should be appreciated that the decision by the occupant to select and purchase any given item in the mini-bar, is not initiated by occupant device 110 and is preferably initiated herein by the consumer reaching towards the mini-bar wherein the mini-bar can recognize the user ID and open the mini-bar automatically for item dispensing.

As therefore may be appreciated, in a ride sharing environment, the intended subscribed rider/user/occupant may search for and select a given vehicle for ride-sharing purposes, and such intended rider may then be supplied on a device with APP a listing of those items and/or promotions that are available in the mini-bar. Such activity may also then include downloading of selected identification information from the occupant device 110 for use by the processor 104. Upon entry into the ride sharing vehicle, the rider may reach towards the mini-bar, which can now confirm the presence of the rider via APP ID (order ride/vehicle and subscription) 106, and the mini-bar automatically unlocks and as the occupant/user approaches the mini-bar the mini-bar opens and the rider can then reach in and select a desired product, which is all now monitored by cameras and sensors 106.

Upon removal of the item the rider's account is charged the appropriate amount by processor 104 and such billing activity is then sent to the rider's smart phone (APP) or occupant device 110. In addition, the mini-bar operation is configured such that if the rider elects to return the unused/unconsumed item the processor 104 (with cameras and sensors recognizing and identifying the activity) may be configured to then credit the rider's account. In addition, the processor may be programmed to restrict such return option by the occupant under selected circumstances, such as within a given time interval or as the system may identify the returned product has been opened, consumed or tampered with via camera recognition and weight sensors.

In other words, the processor 104 with software & trained artificial intelligence or AI is configured such that it preferably includes a wireless transmitter and receiver wherein the processor provides a pre-determined price/cost for each item from said mini-bar transmitted to an external payment system that is external to said mini-bar and which allows said external payment system to wirelessly process payment for said at least one item from said mini-bar to an identified rider/occupant.

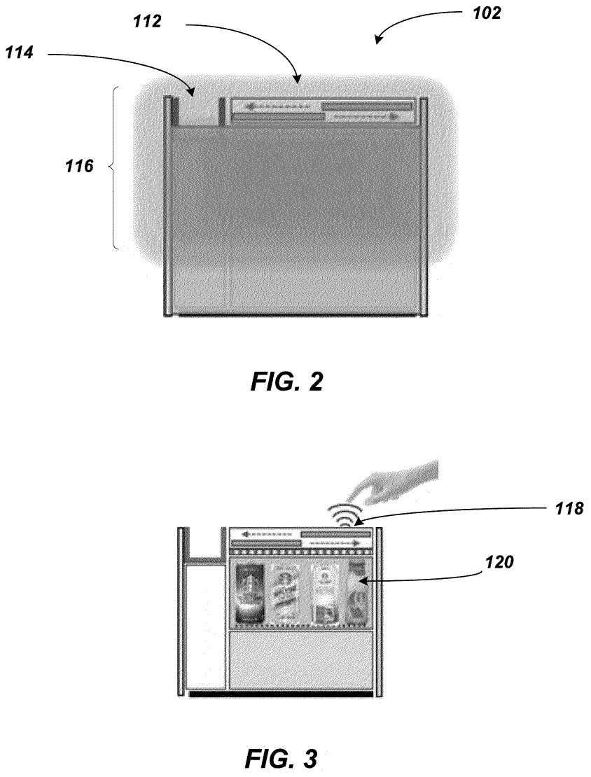

Attention is next directed to FIGS. 2-5 which show in sequence a preferred example of a user accessing and utilizing the mini-bar 102. More specifically, in FIG. 2, one can see the mini-bar 102 in cross-section, which preferably has an automatic door opening capability utilizing a sliding door opening shown at 112. The mini-bar also preferably includes a receptacle 114 which is capable of providing heating/cooling to an inserted item from the mini-bar. The receptacle therefore preferably includes a switch or actuator to allow the user to select either a heating or cooling sequence. Preferably the system itself identifies the item and automatically selects a heating/cooling sequence. As seen at 116, the mini-bar includes UVC lighting which provides safe anti-bacterial lighting and disinfectant lighting as may be directed by the processor 104. Accordingly, it may be understood that mini-bar access may lock and prevent exposure of UVC light to a user, when implementing a selected UVC sterilization protocol.

Preferably, the mini-bar therefore includes one or more LEDs, including LED UVC lighting diodes that may be instructed by the processor to provide disinfection of the mini-bar after access and dispensing of any items therein. Alternatively, the processor may instruct the LED UVC lighting diodes to implement a safe disinfecting lighting sequence at selected interval times and for a selected duration in order to ensure that the contents therein remain in a sterile condition when dispensed to any current or future vehicle occupant.

Expanding on the above, term LED refers to light emitting diodes of all types (including semi-conductor and organic light emitting diodes), and which may be configured to generate light in all or various portions of one or more of the visible, ultraviolet, and UV spectrum. Non-limiting examples of suitable LEDS that may be used include various types of RGB LEDs, addressable RGB LEDs, single color LEDs, infrared LEDS, ultraviolet LEDS, red LEDS, green LEDS, blue LEDS, yellow LEDS, amber LEDS, orange LEDS, and white LEDS. Such LEDS may be configured to emit light over a broad spectrum (e.g., the entire visible light spectrum) or a narrow spectrum.

The LED used in the present disclosure may be formed by one or a plurality of individual LEDs. For example, the LED light source may be configured to include a number of individual LEDs that emit different spectra but which, collectively, emit light that is of a desired color (e.g., white, red, blue, green, yellow, orange, amber, etc.) and/or color temperature (kelvin—symbol shown as K). An LED may also be associated with one or more phosphors that are an integral part of the LED.

The LEDs herein may be independently addressable by the processor 104 for causing emission of light therefrom collectively and/or independently. The term “UVC” when used to describe an LED or the light emitted by the LED means the LED emits light with a dominant wavelength between 200 nm and 280 nm. UVC light is particularly effective in disinfecting the mini-bar inner/interior surfaces and the surfaces of the items/contents from viruses.

Another cross-sectional view of the mini-bar is shown in FIG. 3 where a user has reached toward the mini-bar and a capacitive sensor 118 on the mini-bar detects the presence of the user and then automatically instructs the mini-bar to open without the user touching the surface. However, as alluded to above, the mini-bar herein may also be configured with a manually operated door where the identified user unlocks the door to access the mini-bar items. Alternatively, the mini-bar herein may include a touch sensor which is touched by a user, which touch sensor then can communicate with the processor 104 and in this alternative, preferably identify the user (e.g. fingerprint scan) and allow such identified user mini-bar access. When relying upon the touch sensor, the opening and unlocking of the mini-bar may be permitted to proceed manually or it may be automatic.

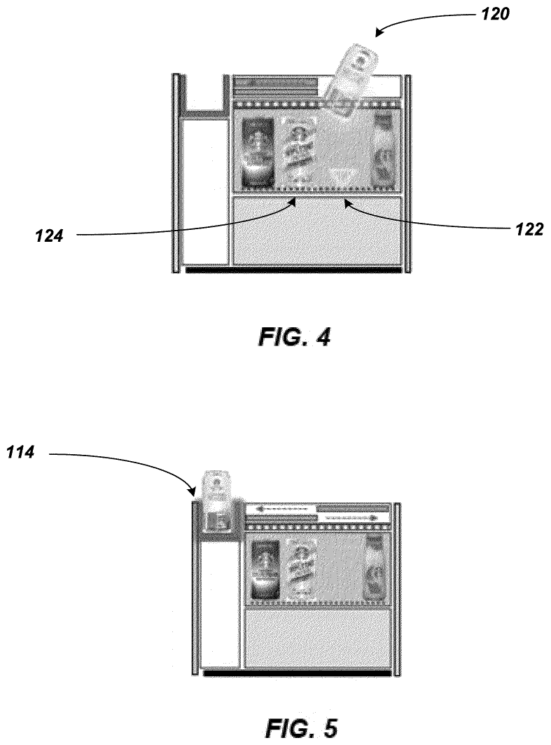

As can be seen, and by way of example only, the mini-bar may contain various beverage items 120 that may be cooled by the mini-bar to a recommended or desired temperature. In FIG. 4, the item is being removed from the open mini-bar. As shown generally at 122, the mini-bar may preferably include a weight sensor that informs the processor 104 with recognition software as to which particular item or items have been removed and should be billed to the identified user occupant. While the mini-bar therefore preferably includes cameras and other sensing devices on the exterior of the minibar to monitor and identify a user, as well as cameras in the interior perimeter surrounding sensing frame 130 to identify an item that is removed, the mini-bar as noted may also include a weight sensor 104 to separately or in combination with other sensors, confirm the dispensing of a selected item. In addition, the mini-bar may include one or more suction devices 124 preferably positioned beneath the items for dispensing, to either stabilize the items within the mini-bar from vehicle motion and/or to restrict or prevent removal of more than one item at a time to a given user.

Attention is next directed to FIG. 5 where the user may position the dispensed item into the receptacle 114 for, as noted herein, heating or cooling. It may be appreciated therefore that once the dispensed item is heated or cooled as desired by the user, the mini-bar may then cycle back to what is illustrated in FIG. 1 and the processor 104 associated with the mini-bar may then launch the UVC sterilization protocol before any additional items are dispensed.

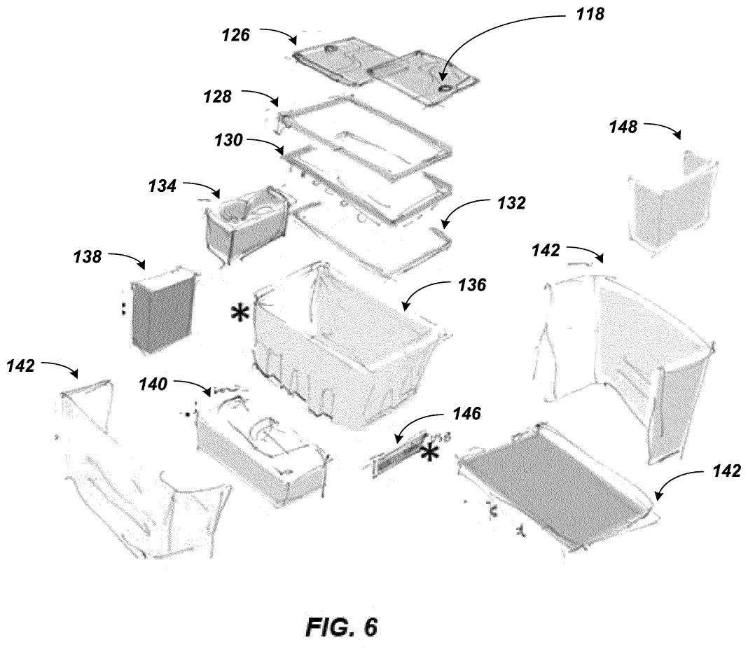

Attention is next directed to FIG. 6 which illustrates in cut-away view a preferred configuration for construction of the vehicle mini-bar herein. Preferably, the mini-bar includes a top sliding portion, of either one or two panels, that includes the previously noted proximity sensor 118 to detect the approach of the user. Such panels may preferably be translucent and formed from a thermoplastic polymeric material or glass (either may be preferably treated with an anti-microbial coating). The mini-bar also preferably includes a frame or liner track 128 and a sensing frame 130 which sensing frame may contain one or a plurality of camera components. Illustrated at 132 is a frame that may supply one or more LED diodes including one or more UVC light emitting diodes. Illustrated at 134 is an inductive (or thermal electric device or Peltier) heating module which may contain one or a plurality of receptacles and at at 136 is an interior cool box which may be preferably made of rigid polymeric foam material (or alternative insulation method) and polymer gladding (surface). At 138 the vehicular mini-bar contains a processor unit that is able and configured to carry-out the herein various functions, as described above in connection with FIG. 1. A compressor or thermoelectric device (or Peltier device) is identified at 140, side panels at 142 and a base attachment panel at 144. Such panels may similarly be preferably sourced from a translucent thermoplastic material, which preferred materials include polycarbonate, polysulfone, and polyetherimide. A USB interface (for charging devices) may be provided at 146. A waste storage bin 148 may optionally be included that is configured to fit on the outside of the vehicle.

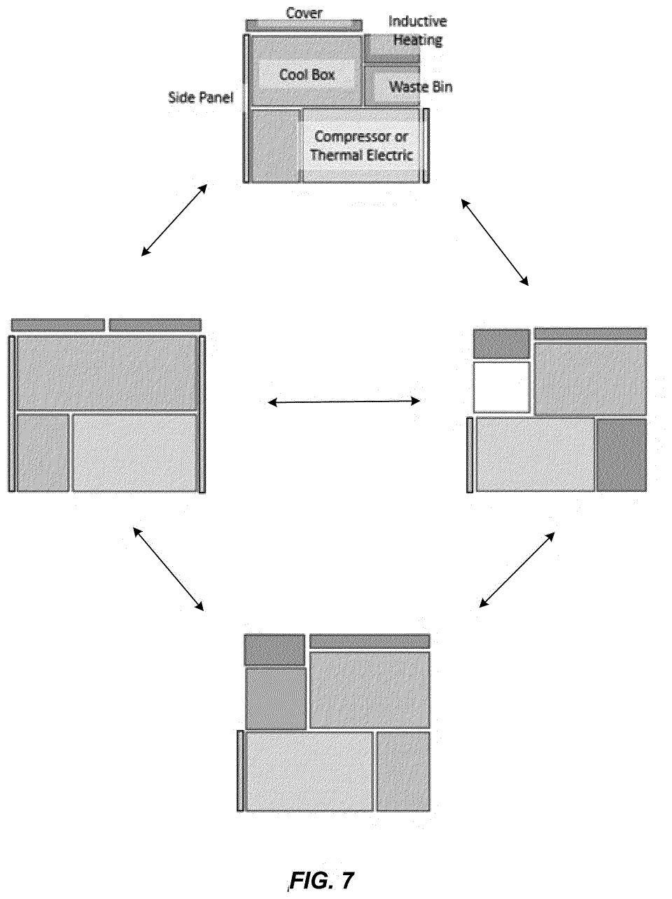

FIG. 7 illustrates the versatility contemplated for the mini-bar herein in cross-section. More specifically, the mini-bar herein may be assembled from various identified components, which therefore allows the mini-bar to adapt different configurations depending upon the needs, e.g., of a given vehicle or vehicle fleet.

As may therefore be appreciated from the above, the vehicle mini-bar herein is one that preferably includes one or more of the following features: (1) controlled temperature environment containing detectable purchase items via optical sensors or load sensor; (2) cameras that are located within the mini-bar that are capable of object recognition, shapes, colors or barcodes; (3) ability to provide occupants with supplemental heating/cooling of selected mini-bar contents; (4) touch free access and APP (e.g. smart phone) supported logistics/delivery/inventory presentation, pricing and billing; (5) built-in UVC disinfectant capability and processor triggered implementation of disinfecting protocols depending upon variables such as time and degree of user access. As noted above, the mini-bar herein is also one that allows for the user/occupant to only touch the item selected for purchase and there is no requirement that the user initiate any such transaction via a separate device, such as a user smart phone device.

Furthermore, the associated software and processor herein is contemplated to manage all activity including logistics, selection and delivery of content/items, inventory and ordering for any given vehicle's mini-bar, and interfaces with the ride service APP, identification/security, purchase, billing, invoicing and returns.

The AI in the software is also contemplated to identify the customer and based on collected data and user profile, may identify preferred and desired items (brands) prior to the ride at the moment the service or destination is selected by the users (APP). The ride operator may then be notified and stock the mini-bar with such preferred item for the identified customer. This then provides a virtual concierge/valet for a personalized experience for selected customers.

The AI and machine learned algorithms can also predict low & high coverage—ride request, as well as segmenting the selection of items/content based on day and time, weather, and local/neighborhood. This may be done automatically with the items delivered and intercepted by the driver or service and the mini-bar restocked accordingly.

External services (shopping/delivery) may also be included in the logistics and use of the secure mini-bar. The user may therefore have carryout food/light groceries or other items delivered to the vehicle prior or in route to their destination.

As also may be appreciated from the above, the present invention relates to a vehicle mini-bar comprising a housing containing one or more items therein for dispensing, preferably a touchless interface configured to detect and allow one or more users to access and purchase a user selected item located within said housing, one or more sensors to identify one or more items contained in said housing and one or more sensors to identify a selected user. A processor is provided in communication with a user device wherein the processor reports to the user device the selection and purchase by a user of said user selected item. The mini-bar also includes one or more UVC light emitting diodes (LEDs) and the processor is configured to activate the one or more UVC LEDs to implement a disinfecting lighting sequence directed at the interior of the mini-bar which is exposed to a given user during access.

The present disclosure is not limited in its application to the details of construction and the arrangement of components set forth in the following description or illustrated in the drawings. The examples described herein may be capable of other embodiments and of being practiced or being carried out in various ways. Also, it may be appreciated that the phraseology and terminology used herein is for the purpose of description and should not be regarded as limiting as such may be understood by one of skill in the art. Throughout the present description, like reference characters may indicate like structure throughout the several views, and such structure need not be separately discussed. Furthermore, any particular feature(s) of a particular exemplary embodiment may be equally applied to any other exemplary embodiment(s) of this specification as suitable. In other words, features between the various exemplary embodiments described herein are interchangeable, and not exclusive.

What is claimed is:

1. A vehicle mini-bar comprising
a housing containing one or more items therein for dispensing;
an interface configured to detect and allow one or more users to access and purchase a user selected item located within said housing;
one or more cameras and sensors to identify one or more items contained in said housing;
one or more sensors to identify a selected user;
a processor with recognition software in communication with a user device (APP) wherein said processor reports to said user device the selection and purchase by a user of said user selected item; and
wherein said mini-bar includes one or more UVC light emitting diodes (LEDs) and said processor is configured to activate said one or more UVC LEDs to implement a disinfecting lighting sequence.

2. The vehicle mini-bar of claim 1 wherein said interface includes a touchless interface.

3. The vehicle mini-bar of claim 1 wherein said interface includes a touch sensor.

4. The vehicle mini-bar of claim 1 wherein said user manually unlocks and opens said mini-bar.

5. The vehicle mini-bar of claim 1 wherein said processor/software implements said disinfecting lighting sequence subsequent to access and removal of a user-selected item.

6. The vehicle mini-bar of claim 1 wherein said processor/software implements said disinfecting lighting sequence at selected time intervals.

7. The vehicle mini-bar of claim 1 wherein said one or more sensors to identify one or more items contained in said housing comprise one or a plurality of cameras.

8. The vehicle mini-bar of claim 1 wherein said one or more sensors to identify one or more items contained in said housing comprises a weight sensor.

9. The vehicle mini-bar of claim 1 wherein said housing provides a controlled temperature environment.

10. The vehicle mini-bar of claim 9 wherein said controlled temperature environment is provided by a compressor or thermoelectric device.

11. The vehicle mini-bar of claim 1 further comprising a receptacle for heating or cooling a user-selected item.

12. The vehicle mini-bar of claim 1 wherein the processor includes a wireless transmitter and receiver wherein the processor provides a cost for at least one item from said mini-bar transmitted to an external payment system that is external to said mini-bar and which allows said external payment system to wireless process payment for said at least one item from said storage unit to an identified user.

13. The mini-bar of claim 1 configured to position within or as part of a vehicle trim component.

14. A vehicle trim component including the mini-bar of claim 1.

15. A vehicle including the vehicle trim component of claim 14.
